(19) United States
(12) Patent Application Publication
(10) Pub. No.: US 2026/0158096 A1
(43) Pub. Date: Jun. 11, 2026

(54) Methods and Formulations for Immuno-Rejuvenation to Prevent or Mitigate Inflammaging Through the Epigenetic Impact of a Polyphenol-Rich Agent Derived from Uniquely Applied Tartary Buckwheat

(71) Applicant: Big Bold Health, Bainbridge Island, WA (US)

(72) Inventors: Jeffrey S. Bland, Bainbridge Island, WA (US); Austin Perlmutter, Bainbridge Island, WA (US)

(21) Appl. No.: 19/402,110

(22) Filed: Nov. 26, 2025

Related U.S. Application Data
(60) Provisional application No. 63/728,948, filed on Dec. 6, 2024.

(51) Int. Cl. A61K 36/70; A61K 31/137; A61K 31/19; A61K 31/352; A61K 31/375; A61K 31/555; A61K 31/7048; A61K 33/06; A61P 29/00

(52) U.S. Cl. CPC A61K 36/70; A61K 31/137; A61K 31/19; A61K 31/352; A61K 31/375; A61K 31/555; A61K 31/7048; A61K 33/06; A61P 29/00; A61K 2236/11; A61K 2236/15; A61K 2236/19

(57) ABSTRACT

Development of producing certain methods of and nutrition management of Tartary buckwheat (Fagopyrum Tataricum) to promote immuno-rejuvenation by influencing key pathways involved in aging, inflammation and immune system function.

### CROSS-REFERENCE TO RELATED APPLICATIONS

This application has priority from U.S. Provisional Patent Application No. 63/728,948 filed Dec. 6, 2024.

### GOVERNMENT SUPPORT

No support of the United States government was made in the conception, development or reduction to practice of the present invention.

### FIELD OF THE INVENTION

This invention relates to the development of nutraceutical agents designed to modulate immune system aging through epigenetic mechanisms. Specifically, it describes a polyphenol-rich agent derived from Tartary buckwheat (Fagopyrum tataricum) that promotes immuno-rejuvenation by influencing key pathways involved in aging, inflammation, and immune system function.

This invention enhances the utilization of Tartary buckwheat and its unique nutrient profile to mitigate inflammaging-a is aimed at reducing age-related inflammatory processes through the bioactive compounds present in Tartary buckwheat. Tartary buckwheat is recognized for its rich composition of bioactive compounds, including polyphenols, flavonoids (such as rutin and quercetin), dietary fiber, and essential amino acids. These components have been associated with various health benefits through antioxidant, anti-inflammatory, metabolic, epigenetic and other effects. The application of Tartary buckwheat in combating inflammaging leverages its potential to modulate key pathways which may help slow the aging process and decrease risk for related chronic diseases. But there is a need for enhancements through new understanding particularly in rejuvenation of biological age. The present invention provides such understanding, implementation and the utility thereof.

### BACKGROUND

Aging is associated with a decline in immune function, known as immunosenescence, and increased chronic inflammation, referred to as “inflammaging.” These processes result in susceptibility to infections, autoimmune disorders, and decreased vaccine efficacy. There is also increasing evidence that alterations in immune function are associated with chronic conditions such as chronic fatigue, mood disorders, cognitive impairment, chronic pain, sleep disturbance, poor exercise tolerance, metabolic disorders including insulin resistance and obesity, preclinical autoimmune disorders, skin problems, and digestive disorders. Emerging research indicates that specific dietary polyphenols-compounds uniquely abundant in Tartary buckwheat—have significant impacts on the biology of the immune system and its relationship to age-related disorders by influencing epigenetic regulation, including DNA methylation, histone modification, and chromatin remodeling. Recent studies have demonstrated that dietary polyphenols:
1. Reduce oxidative stress and chronic inflammation.
2. Rejuvenate immune cell function by restoring youthful epigenetic profiles.
3. Influence pathways involved in autophagy, proteostasis, and mitochondrial health, key determinants of cellular aging.

Notably, the unique variety of Tartary buckwheat (i.e. Himalayan Tartary buckwheat) and simulations shown herein used in this embodiment contains more than 126 polyphenols and flavonoids such as rutin, quercetin, and other potent flavonoids that exhibit high bioavailability and bioactivity, making it an ideal candidate for addressing immunosenescence and promoting longevity.

A detailed immune epigenetic research investigation of Himalayan Tartary buckwheat concentrate is provided in the publication Perlmutter et al. Frontiers of Nutrition 2024, 18 Nov., 2024 and discussed herein. The insights of the present application go beyond those of the publication and a further clinical trial is being made to further advance scopes.

### Definitions

Buckwheat—is a seeded flowering plant (not grain nor rice) long used as a pseudo-cereal. It has varieties of common buckwheat (Fagopynum esculentum) and tartary buckwheat (Fagopyrum tataricum) reflecting earlier recognized use in the tartary Mongolian) region, a difficult growth region.

CpG Islands—Genomic clusters (islands of cytosine followed by guanine on a nucleotide strand) of at least 200 base pairs long with high (50/+) G&C content in DNA involved in genome expression control.

DNA Demethylation—Removal of methyl group from DNA facilitated by enzymes.

Epigenetic age clocks—are biochemical tests used to measure biological age using a person or domesticated animal subjects DNA methylation level changes at CpG dinucleotide to establish effectiveness of anti-aging interventions as to methyl group attachments to DNA

Gene Ontology (GO)—A knowledge-base framework for describing functions of gene products enabling analysis of molecular biology through use of strand concepts.

Inflammation—is a body response to injury/invasion promoting healing through immune system responders' reaction to inflammatory cells and cytokines, but also itself a danger to a body when triggered by means other than injury/incursion (chronic inflammation).

### SUMMARY OF THE INVENTION

This invention disclosure includes isolation methods and formulations of a novel polyphenol-rich agent derived from Tartary buckwheat that serves as a functional food or nutraceutical for immuno-rejuvenation. The agent is formulated to modulate epigenetic mechanisms, including DNA methylation patterns and histone acetylation, to restore immune function and reduce inflammaging.

This invention disclosure encompasses:
1. Isolation Techniques: Methods for isolating and concentrating immune bioactive ingredients from Tartary buckwheat.
2. Formulation Approaches: Development of capsules, powders, or functional beverages to deliver the agent effectively.
3. Mechanistic Insights: Evidence demonstrating the epigenetic and immunological impact of the agent in human and preclinical studies.

The key components of Tartary buckwheat that contribute to its anti-inflammatory and anti-aging effects include:

### 1. Rich Polyphenol Content

Tartary buckwheat carriage of polyphenols, particularly rutin and quercetin in Tartary buckwheat seeds contain approximately 0.5-1.4% dry weight of rutin, markedly higher than common buckwheat seeds. These compounds play a crucial role in:
neutralizing free radicals to reduce oxidative stress, a major contributor to inflammaging
modulating inflammatory pathways, such as the NF-KB pathway, which is often overactivated in aging populations.
reducing levels of pro-inflammatory cytokines, such as interleukins (IL-6, IL-113) and tumor necrosis factor-alpha (TNF-a).
influencing epjgenetjc pathways related to inflammaging
beneficially influencing metabolic pathways that modulate inflammaging, including supporting lipid metabolism, which helps mitigate chronic inflammation caused by dyslipidemia.

### 2. High Levels of Immune-Active Vitamins and Minerals

Tartary buckwheat content includes vitamins and minerals that play pivotal roles in mitigating inflammaging, the chronic, low-grade inflammation. Its content of vitamin E, a powerful antioxidant, helps combat oxidative stress, a key driver of inflammaging that neutralizes free radicals and also modulates inflammatory pathways by reducing the production of pro-inflammatory cytokines such as IL-6 and TNF-α. Additionally, Tartary buckwheat provides significant levels of vitamin B6, which is critical for the synthesis of neurotransmitters and cytokine regulation, helping to maintain immune balance and reduce the chronic inflammation often seen in aging populations.
The mineral composition of Tartary buckwheat further supports its anti-inflammaging potential. Zinc, a key trace element, regulates the activity of immune cells and has been shown to decrease markers of systemic inflammation, such as C-reactive protein (CRP). It also plays a role in repairing damaged DNA and maintaining telomere stability, processes crucial for slowing age-related immune decline. Lower levels of zinc have been explicitly linked to immune aging in preclinical research. Magnesium, another essential mineral in Tartary buckwheat, helps regulate the body's stress response and inhibits the activation of inflammatory pathways like NF-KB. Moreover, the presence of selenium, though in trace amounts, enhances antioxidant defenses by supporting glutathione peroxidase activity, further reducing oxidative damage and chronic inflammation. Together, these nutrients in Tartary buckwheat target the root mechanisms of inflammaging, offering potential benefits for healthy aging and immune resilience.

### 3. Anti-Inflammatory Dietary Fiber and Unique Carbohydrates

Tartary buckwheat is also an excellent source of dietary fiber, offering both soluble and insoluble fibers (including resistant starch) that contribute to gut health, microbiome wellness and potentially mitigate inflammaging. Soluble fiber in Tartary buckwheat acts as a prebiotic which may promote the growth of beneficial gut bacteria such as Lactobacillus and Bifidobacterium. These microbes ferment the fiber to produce short-chain fatty acids (SCFAs) such as butyrate, acetate, and propionate, which have been shown to exhibit potent anti-inflammatory effects. SCFAs modulate immune responses by inhibiting the activation of inflammatory pathways like NF-KB and reducing the production of pro-inflammatory cytokines, thereby addressing one of the root causes of inflammaging. Insoluble fiber in Tartary buckwheat plays a critical role in maintaining intestinal integrity, which is vital for preventing systemic inflammation. By promoting regular bowel movements and reducing intestinal permeability, insoluble fiber helps prevent the leakage of endotoxins like lipopolysaccharides (LPS) into the bloodstream-a key trigger of systemic inflammation in aging populations. Additionally, fiber can influence the gut-brain axis, potentially alleviating neuroinflammation linked to cognitive decline in older adults. The combined prebiotic and gut-protective properties of Tartary buckwheat's dietary fiber make it a valuable component of a diet aimed at reducing inflammaging and supporting healthy aging. In addition to dietary fiber, Tartary buckwheat contains the unique carbohydrate D-chiro-inositol (DCI), a sugar with known insulin-like activity. Beyond the metabolic benefits of DCI that may translate into decreased risk for inflammaging, DCI is also recognized to have beneficial effects on decreasing inflammation, with in vitro studies demonstrating a suppression of multiple pro-inflammatory factors.

### 4. Unique Amino Acid Composition

Tartary buckwheat is rich in essential amino acids and contains essential amino acids. It is particularly rich in leucine. Adequate consumption of amino acids is critical for preventing age-related muscle loss (sarcopenia) which can contribute to inflammaging. Tartary buckwheat's synergistic effects on inflammation, oxidative stress, and metabolic health make it a powerful natural intervention for managing inflammaging. Its nutrient profile may directly address the underlying mechanisms of inflammaging, including:
delaying the onset of senescent cells that drive chronic inflammation.
balancing immune responses to prevent excessive inflammation.
protecting against degenerative changes in tissues and organs.

By integrating Tartary buckwheat into functional foods, dietary supplements, or nutraceutical products, humans and some other lines of mammals can effectively combat inflammaging and promote healthy aging. This provides a practical, natural, and sustainable approach to addressing significant challenges of aging populations.

### DRAWING FIGURES

FIG. 1 labeled “Chromosomes” shows a Manhattan Plot showing visual representations of data (differentially methylated loci (DML)) including chromosound locations and optical distribution for an epigenome-wide analysis identifying CpGs related to intake of Tartary buckwheat extract in the study of the above mentioned Nov. 18, 2024 publication. It depicts genes associated with their vertical position corresponding to the negative logarithm (base 10) of the unadjusted p-value for DNA methyl association with significance threshold set at p=0.001. The x-axis shows genomic positions organized by chromosomes with color shading (color versions attached to this application in Appendix A) with shaded/colored-coded dots indicating specific chromosomes, with different shades of blue (in colored version) sized to demonstrate separate chromosomes. Prominently peaked dots represent CpG AI that surpass the genome wide significancy threshold, indicating significant associations.

FIG. 2 labeled Differential Methylation shows a Volcano Plot for epigenome-wide association study and enrichment analysis. The plot illustrates differentially methylated loci (DML) identified in a pre-vs. post-intervention comparison. It is black and white as herein shown, but implemented by a colored version in Appendix B hereof. Each dot represents a CpG site with its vertical position indicating the negative logarithm (base 10) of the unadjusted p-values for DNA methylation association. The x-axis shows the relative log fold change (log FC) of the m-values between the two timepoints. Negative values indicate CpGs with decreased methylation among study participants (green and white positive values indicate increased methylation (red)).

FIGS. 3 and 4 have three tiers-A, B, C indicating gene ontology, GO-BP (biological process) at A, GO-MF (molecular function at B) GO-CC (cellular component). FIG. 3 is from hypermethylation and FIG. 4 is for hypomethylation

FIG. 5 is a Venn diagram of DMLs, identified between compositions of a Dwarka et al. (TruDiagnostics, Inc.) study at BMC Medicine (2024) 22:30 https//doi.org/10.1186/s12916-024-03513-w plotted against DMLs from a Tartary buckwheat supplemental intervention.

FIG. 6 is a visual representation of individual epigenetic age accelerations.

### DETAILED DESCRIPTION OF THE INVENTION

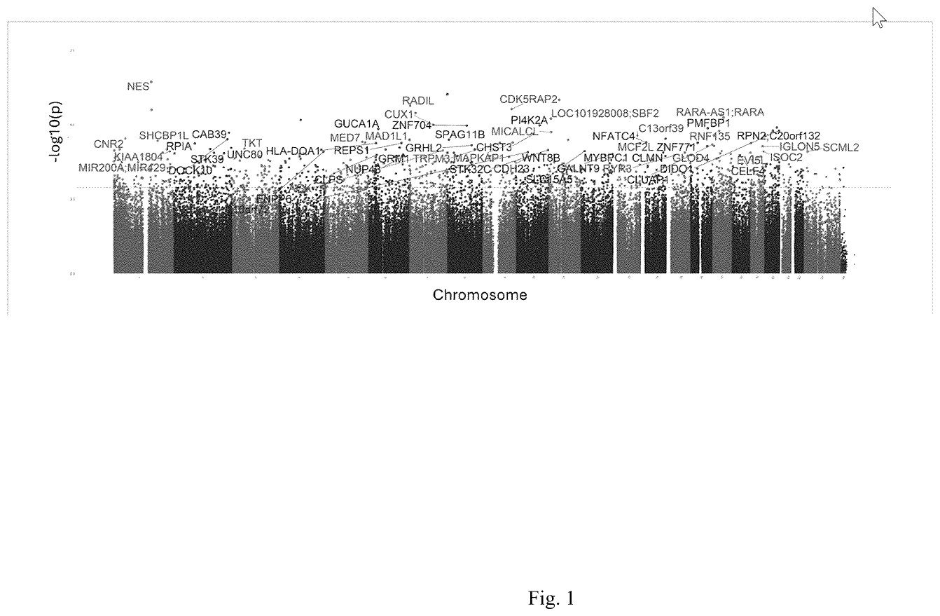

A comprehensive analysis of the antioxidant and polyphenol makeup of Tartary buckwheat using the below described unique methodology demonstrates a signature of molecules specific to this farming and sprouting technology. (See FIG. 1). Tartary buckwheat's bioactive compounds exert a multifaceted influence on key biological mechanisms implicated in inflammaging, including the reduction of oxidative stress, modulation of inflammatory pathways and metabolic benefits. The interplay of these mechanisms underpins its effectiveness as a natural intervention for promoting healthy aging.

### Reducing Oxidative Stress and Inflammation

### Reduction of Oxidative Stress:

Tartary buckwheat's high concentration of antioxidants, including flavonoids (e.g., rutin and quercetin) and polyphenols target and neutralize reactive oxygen species (ROS), which are central drivers of cellular damage and inflammaging. By preventing or mitigating the effect of oxidative damage to DNA, proteins, and lipids, these compounds may reduce the chronic activation of pro-inflammatory pathways triggered by oxidative stress. Studies show that the polyphenols in Tartary buckwheat, including rutin and quercetin have antioxidant capabilities.

### Anti-Inflammatory Effects:

Tartary buckwheat's polyphenols suppress the production of pro-inflammatory mediators, such as cytokines and chemokines, while promoting the resolution of inflammation. This dual action helps alleviate chronic, low-grade inflammation that is characteristic of aging.
Our research highlights that Tartary buckwheat extract can effectively suppress inflammatory mediators like tumor necrosis factor-a, interleukin-(IL-) 6, and IL-12, while a polyphenol extract decreased protein levels of inflammatory mediators including myeloperoxidase, iNOS, COX2, IL-6, IL-1B. and TNF-a.

### Modulation of the NF-κB Pathway:

The NF-κB signaling pathway is a pivotal regulator of inflammation and is often hyperactivated in inflammaging. Tartary buckwheat polyphenols, particularly rutin and quercetin, may inhibit NF-κB activation by suppressing upstream signals such as IKB kinase (IKK) and preventing the nuclear translocation of NF-κB. This reduces the transcription of pro-inflammatory cytokines and enzymes, thereby mitigating systemic inflammation. An extract from Tartary buckwheat has been found to suppress NF-κB in macrophages, while polyphenols from Tartary buckwheat have a similar effect in hepatic cells.

### Suppression Pathways

### Suppression of Metabolic Pathways Linked to Inflammation:

Metabolic dysfunction is known to have a bidirectional effect on inflammation and inflammaging. Tartary buckwheat extracts have been found to block metabolic dysfunction at a cellular level as well as to improve metabolic parameters therefore decreasing inflammatory activation and inflammaging pathways an extract from Tartary buckwheat has been found to suppress unhealthy metabolic activation in adipose cells, which tethers to decreased inflammatory activation. In a high-fat diet fed mice, Tartary buckwheat prevented both metabolic disturbances and inflammation.

### 1. EXAMPLES OF FORMULATIONS

Tartary buckwheat can be utilized in a variety of formulations designed to maximize its bioactive potential while catering to consumer preferences and specific health needs. Examples include:

Powders: Finely milled Tartary buckwheat powder can be incorporated into smoothies, soups, or baked goods. Enhanced formulations may include additional ingredients like probiotics, prebiotics, or complementary antioxidants for synergistic health benefits.

Capsules and Tablets: Standardized extracts of Tartary buckwheat rich in polyphenols (e.g., rutin, quercetin) can be encapsulated or compressed into tablets. Ideal for individuals seeking a convenient, controlled dosage of bioactive compounds.

Ready-to-Drink Products: Beverages infused with Tartary buckwheat extracts, such as teas, functional drinks, or meal replacement shakes, provide a quick and easy way to consume its nutrients and preferable fortified with additional nutrients (e.g., vitamins C and E) to boost antioxidant capacity.

Functional Food Products: Tartary buckwheat flour can be used in the production of crackers, granola bars, and breakfast cereals incorporation into fermented foods like yogurt or kombucha can enhance bioavailability and gut health benefits.

Topical Products: Using techniques like solvent extraction, microwave-assisted extraction, and pulsed electric field extraction, key elements of Tartary buckwheat can be extracted for use in topical products for skin application.

### 2. DOSAGE FORMS AND RECOMMENDED INTAKE

The recommended intake of Tartary buckwheat bioactive components depends on the formulation and health goal. General guidelines include:

Powders: 6-24 grams daily of sprouted corrected to powder, or 20 grams of flour mixed used in food or beverages at usage of approximately 100-1000 mg of polyphenols per diem, depending on source.

Capsules/Tablets: 500-2,000 mg of standardized extract per day, divided into 1-2 doses, designed to deliver a consistent dose of key compounds found in Tartary buckwheat of rutin and quercetin and like components.

Ready-to-Drink Products: 250-500 ml per day, delivering 50+ mg of polyphenols per serving. Ideal for on-the-go consumption.

Topical Products: delivering key nutrients from Tartary buckwheat directly to the skin.

These dosages are based on existing studies and can be adjusted based on individual needs and the concentration of active compounds in the formulation.

### Target Demographics and Conditions

Tartary buckwheat-based products are well-suited for a range of demographics and health conditions, including:

Aging Populations: Individuals over 50 looking to reduce chronic inflammation and oxidative stress to promote healthy aging and prevent age-related diseases.

Individuals with Metabolic Syndrome: Tartary buckwheat can aid in managing conditions like insulin resistance, dyslipidemia, and hypertension by improving metabolic parameters and reducing inflammation.

Athletes and Active Individuals: The antioxidant and anti-inflammatory properties of Tartary buckwheat can aid in recovery from exercise-induced stress and inflammation.

Health-Conscious Consumers: People seeking natural, plant-based solutions to maintain overall health and wellness.

Specific Health Conditions: Those with inflammatory disorders (e.g., arthritis, autoimmune diseases).

Individuals at risk of cardiovascular or neurodegenerative diseases due to inflammaging.

### 3. ACTIVE COMPONENTS AND MECHANISMS OF ACTION

Polyphenol-rich formulations according to the present disclosure were obtained by isolation of active ingredients from Himalayan Tartary buckwheat and Tartary Buckwheat Simulation of the Himalayan Tartary Buckwheat and further enhanced by combination with synergistic compounds including hydroxymethylbutyrate. Such formulations include:
126 polyphenols and flavonoids found in Himalayan Tartary buckwheat including, but not limited to:
   Rutin: A bioflavonoid shown to reduce inflammation and oxidative stress through NF-κB modulation and SIRT1 activation.
   Quercetin: A flavonol that promotes autophagy and protects against immune cell DNA damage.
   Luteolin: A flavonoid recognized to support immune function
   Hesperidin: A flavonoid recognized to serve as a redox signaling agent in immune cells
D-chiro-Inositol: A compound with potential roles in metabolic regulation and immune health.

Agent efficacy is mediated through:
Epigenetic Modulation: The agent restores youthful DNA methylation patterns in immune cells, enhancing their functionality.
Reduction of Chronic Inflammation: By inhibiting pro-inflammatory cytokines such as IL-6 and TNF-α, the agent mitigates inflammaging.
Promotion of Cellular Health: Polyphenols improve mitochondrial function and proteostasis, essential for maintaining cellular homeostasis during aging.

### 4. PREPARATION AND FORMULATION

Formulations as disclosed herein are derived from Tartary buckwheat using eco-friendly organic and regenerative agriculture production methods followed by processing to concentrate polyphenols while preserving bioactivity. Such formulations may be enriched with synergistic compounds such as omega-3 fatty acids and specific probiotic organisms and other nutrients to enhance its immunomodulatory properties.

Sustainable Cultivation: To ensure the preservation of bioactive components and minimize environmental impact, we have adopted sustainable agricultural practices, including regenerative and organic farming. The combination of these technologies and techniques describes a vertically integrated model of nutrient optimization to maximize anti-inflammaging effects of finished products.

Regenerative Farming: crop rotation, cover cropping, and minimal tillage to enhance soil health and biodiversity. Regenerative methods to improve soil organic matter, reduce erosion, and increase the availability of nutrients critical for the growth of nutrient-dense Tartary buckwheat.

Organic Farming: avoiding synthetic fertilizers and pesticides and promoting natural ecosystems and reducing chemical residues in the final product. Organic methods support the integrity of the crop, ensuring a clean-label product aligned with consumer preferences.

Integration with Local Ecosystems: Tartary buckwheat is well-suited for cultivation in marginal soils and diverse climates, making it a sustainable option for smallholder farmers and underutilized agricultural regions. Its short growing cycle and adaptability allow for efficient land use and water conservation.

Soil-Microbiome Enhancement through Microbial Inoculation: To further optimize nutrient density and preserve the bioactive profile of Tartary buckwheat, the application of seed inoculation with soil-enhancing microbes is described:

Microbial Inoculation: Treating seeds with a mixture of beneficial microorganisms, such as rhizobacteria and mycorrhizal fungi, to enhance nutrient uptake and plant resilience. These microbes improve the availability of key minerals (e.g., zinc, iron) and bioactive precursors in the soil, enriching the nutrient profile of the harvested grain. Inoculation also enhances the plant's resistance to environmental stress, reducing reliance on external inputs.

Microbiome and Bioactivity: Enhancement of soil microbiomes to stimulate the synthesis of secondary metabolites such as polyphenols (e.g. rutin and quercetin) in the plant, increasing its functional health benefits. These techniques align with sustainable agriculture goals by reducing the need for synthetic inputs and improving crop quality.

Sprouting to Enhance and Alter Nutrient Content: Sprouting is an innovative post-harvest processing technique that significantly enhances and modifies the nutrient profile of Tartary buckwheat, particularly for use in sprouted flour:

Sprouting Process: Tartary buckwheat seeds is soaked in water to initiate germination and activation of enzymatic processes that break down antinutritional factors and enhance nutrient bioavailability. Controlled sprouting conditions (e.g., temperature, humidity) are optimized to maximize the synthesis of bioactive compounds. Sprout harvesting occurs at the 3 day mark, consistent with existing research on maximization of polyphenol content (See Chart 2)

Nutrient Alterations: Increase the concentration of flavonoids, particularly rutin and quercetin, as well as essential amino acids such as lysine by sprouting, i.e., harvesting products at sprout cessation.

Sprouted Flour Production: After germination and sprouting, the dried, sprouted seeds are then milled into a fine flour, which can be used in functional food products such as baked goods, nutritional bars, and beverages.

### Enhancing Bioavailability and Potency

To maximize the health benefits of Tartary buckwheat and its bioactive compounds, advanced processing techniques are essential. These methods enhance the bioavailability, potency, and stability of key nutrients, such as flavonoids, polyphenols, and amino acids, ensuring their effectiveness in combating inflammaging.

Micronization: Micronization involves reducing the particle size of Tartary buckwheat or its extracts to the micrometer scale, significantly improving the dissolution and absorption of bioactive compounds:
Mechanism: By decreasing the particle size, the surface area of the material is increased, enhancing its solubility and interaction with digestive enzymes. This process ensures more efficient gastrointestinal absorption of key nutrients like rutin and quercetin.
Benefits: Enhancing bioavailability of poorly water-soluble compounds such as flavonoids. Improved uniformity and dispersibility of Tartary buckwheat-based formulations in powders, beverages, and other products.
Applications: Micronizing Tartary buckwheat powder can be used in capsules, tablets, or functional food products to deliver a higher bioactive payload in smaller servings.

Fermentation: Fermentation is a natural bioprocess that uses microorganisms to transform Tartary buckwheat into more bioavailable and potent forms:
Mechanism: Fermentation with beneficial microbes (e.g., Lactobacillus, Aspergillus, or Saccharomyces species) breaks down complex molecules into smaller, more bioactive forms. For example, rutin is converted into quercetin during fermentation, increasing the availability of this potent antioxidant and anti-inflammatory compound.
Benefits: Enhanced Nutrient Bioavailability: Fermentation reduces antinutritional factors (e.g., phytic acid) that hinder mineral absorption. Microbial activity often boosts the total polyphenol content, enhancing antioxidant capacity. Fermented products provide probiotics and prebiotic metabolites, which support a healthy gut microbiome and systemicanti-inflammatory effects.
Applications: Fermented Tartary buckwheat can be used to create probiotic-rich functional foods, such as yogurts, fermented beverages, or baking ingredients. The process can also be employed to produce high-potency dietary supplements.

### Combined Techniques

Combining micronization and fermentation offers synergistic benefits:
Micronized Fermented Flour: Fermentation followed by micronization creates a fine powder with enhanced nutrient bioavailability and functional properties, ideal for incorporation into a wide range of health products.
Post-Fermentation Encapsulation: After fermentation, bioactive compounds can be encapsulated using lipid or polymer-based carriers to further protect them from degradation and improve targeted delivery.

Formulation 1 was a polyphenol-rich supplement (HTP Rejuvenate) with composition shown in Table 1. HTP Rejuvenate was prepared.

TABLE 1
Formulation 1

| | Amount per | | Amount per | |
|---|---|---|---|---|
| | Serving (2 | | day (4 | |
| Component | capsules) | | capsules) | |
| Himalayan Tartary buckwheat | 95 | mg | 190 | mg |
| (HTB) flour | | | | |
| D-chiro inositol | 150 | mg | 300 | mg |
| (DCI/D-chiro-inositol) | | | | |
| 2-hydroxybenzylamine (2- | 13 | mg | 26 | mg |
| HOBA) | | | | |
| Hydroxymethylbutyrate | 69 | mg | 138 | mg |
| (HMB) | | | | |
| Chlorophyllin | 7.5 | mg | 15 | mg |
| Polyphenols* | 579 | mg | 1,158 | mg |
| Vitamin C | 20 | mg | 40 | mg |
| Calcium | 30 | mg | 60 | mg |

*Polyphenols delivered in one serving (2 capsules) included 330 mg quercetin, 83 mg rutin, 83 mg hesperidin, and 83 mg luteolin.

1. Epigenetic Effects: Studies using primary human immune cells demonstrated that the agent reversed age-related hypermethylation in genes associated with inflammation and immune response.
2. Reduction in Inflammaging: Animal models treated with the agent showed significant reductions in systemic inflammation markers, including C-reactive protein and IL-1B.
3. Human Trials: In a pilot study, older adults consuming HTP Rejuvenate for 12 weeks exhibited improved vaccine responses and reduced inflammatory cytokine levels. Details of the study are provided in the publication Perlmutter et al. Frontiers of Nutrition 2024, 18 Nov., 2024 . . . . Key features of this study are as follows:

### Participant Recruitment and Eligibility:

50 generally healthy (defined as the absence of exclusion criteria below) men and women between the ages of 18 and 85 years (inclusive) with body mass index (BMI)<40 kg/m² were enrolled in the study. This age range was selected to capture a diverse range of adult participants while avoiding the potential for outlier variability introduced at extremes of aging, and to capture the population most likely to be taking a nutritional supplement. Participants were required to have an established primary care provider and active health insurance; be able to read, write, and speak English fluently; and be able to comply with the protocol instructions including performing the in-home venous blood draw using a Tasso device. Investors or immediate family members possessing investment in Big Bold Health were excluded from the study. Women who were pregnant and/or lactating and individuals on jobs requiring night shift work were excluded. Exclusion criteria also included history (prior 2 years) or presence of cancer, except for non-melanoma skin cancer; known history of blood dyscrasias including coagulopathy or use of prescription anticoagulants; diagnosis of a transient ischemic attack (within 6 months); presence of clinically significant acute or unstable cardiovascular or cerebrovascular disease, psychiatric disorder, alcohol or chemical dependence; immune-related conditions (e.g., hepatitis C, HIV, or active infection within the previous 4 weeks) or other illness that in the opinion of the Clinical Investigator would render a participant unsuitable to participate in the study. In addition, those with known allergy to any of the components of the test product, those consuming known prescription immunomodulating products (e.g., oral glucocorticoids, TNF-α inhibitors) or concentrated polyphenolic supplements within 1 month the baseline visit. Concentrated polyphenolic substances that were specifically excluded prior to, and during the study were quercetin, rutin, luteolin, epigallocatechin gallate (EGCG), resveratrol, curcumin, fisetin, berberine, soy isoflavones (genistein, daidzein, and glycitein), hesperidin, and ellagic acid.

### Study Objectives

The primary objective of that exploratory clinical trial was to evaluate the effect of consuming a polyphenol-rich supplement largely based around the phytochemical composition of Tartary buckwheat (HTB Rejuvenate) for 90 days on epigenetically-measured immune age. The secondary objective was to assess the effects of HTB Rejuvenate on peripheral leukocyte immune profiles after 90 days, as well as on GO pathways. Tertiary objectives included capture and review of descriptive clinical observations using a General Health Questionnaire (GHQ). Safety was also assessed via reports of adverse events (AEs).

### Analysis Populations

A review of compliance and protocol deviations was conducted prior to data analysis. Based on the completion of the blood sampling for the epigenetic tests, the modified intent to treat (ITT) population, which includes all participants who completed both baseline and final labs, was composed of n=47. One participant withdrew consent at visit 2, and two participants who did not complete the final blood draw were excluded from the ITT population because the outcome required both tests for analysis. In addition to removal of the two individuals who were excluded from the ITT, the per-protocol (PP) population excluded seven other participants, including two for use of excluded medications/supplements, four for low study supplement compliance, and one for both inclusion of an excluded medication/supplement and low compliance. The final analyzed population therefore included 40 people.

### Demographics

The demographics obtained during screening/baseline clinical interviews for the ITT and PP populations are provided in Table 2. Participants in the PP population, which was the primary population for the laboratory analyses, were 54 y (SD, 11 y) old with BMI of 24.2 kg/m² (SD, 3.3 kg/m²). A total of 14 participants had documented cases of COVID during the study.

TABLE 2
Participant Characteristics

| Characteristic | Units | ITT | | PP | |
|---|---|---|---|---|---|
| Population Number | N | 47 | | 40 | |
| Age | y (±SD) | 54 | (±11)^{‡} | 54 | (±11)^{§} |
| Sex | % Female | 60 | | 62.5 | |
| Weight (self-report)† | kg | 70.3 | (±15.7)^{‡} | 69.2 | (±14.7)^{§} |
| Height (self-report)† | cm | 166.7 | (±11.1)^{‡} | 166.5 | (±11.2)^{§} |
| BMI | kg/m² | 24.6 | (±3.5) | 24.2 | (±3.3) |

BMI, body mass index; cm, centimeter; ITT, intent-to-treat; kg, kilogram; m, meter; N, subject number; PP, per protocol; SD, standard deviation; y, years.
*The ITT data excluded the two participants who did not complete final lab tests.
′Self-reported weight and height data were converted to metric units for reporting purposes.
′The ITT population had no data for 3 participants for age, and 11 participants for weight and height. All participants had reported BMI data.
The PP population had no data for 3 participants for age, and 7 participants for weight and height. All participants had reported BMI data.

### Epigenetic Age Biomarkers

Blood samples were obtained by participants at baseline and after 90 days of starting the trial using an in-home Tasso device and shipped for analysis (TruDiagnostic laboratory). Overall, we analyzed blood samples from 47 adults from an initial sample set of 50 individuals using an Illumina EPICv1 epigenetic panel that analyzed DNA methylation at 850,000 CpG sites prior to and after 90 days of an intervention with a polyphenol-rich supplement designed to mimic major bioactive nutrients found in the plant Tartary buckwheat. Immune assessments were obtained via deconvolution algorithms of the epigenetic data, which quantitatively approximate immune cell subsets including CD4+ T cells, CD8+ T cells, granulocytes, natural killer cells, monocytes, eosinophils, and neutrophils.

Epigenome-Wide Analysis Identifies CpGs Related with Intake of Tartary Buckwheat Extract

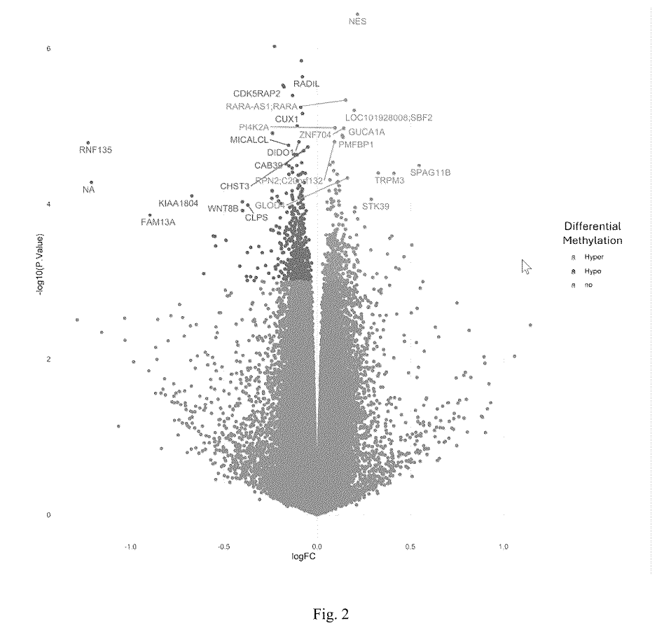

To investigate the overall epigenetic impact of the standardized polyphenol concentrate upon the cohort, the present inventors conducted an epigenome-wide analysis (EWAS) analysis to identify CpGs which showed significant differential methylation between the two visits. From the indicated model, we identified 887 Differentially Methylated Loci (DMLs) across the EPIC/850 K data (unadjusted p-value<0.001). Among these, 336 CpG sites were hypermethylated at the conclusion of the study (Visit 6), while 551 loci were hypomethylated. The results of the analysis are represented in the following Manhattan (FIG. 1) and Volcano plots (FIG. 2) to allow for visual representation of data. The Manhattan plot is used to identify chromosomal locations of the DMLs and to observe whether DMLs were spatially distributed in clusters or across the genome, whereas the Volcano plot allows for visualization of direction of methylation change (e.g., hyper-vs. hypo-methylation).

As noted above, the Manhattan plots of FIG. 1 depict genes associated with CpG sites identified in the analysis. Each dot on the plot represents a CpG site, with its vertical position corresponding to the negative logarithm (base 10) of the unadjusted p-value for DNA methylation association, with a significance threshold set at p=0.001. The x-axis shows genomic positions organized by chromosomes, with color-coded dots indicating specific chromosomes; different shades of blue are used to demarcate separate chromosomes. The prominently peaked dots represent CpG sites that surpass the genome-wide significance threshold, indicating significant associations.

As noted above, the Volcano plot of FIG. 2 illustrates differentially methylated loci (DMLs) identified in the pre-vs. post-intervention comparison. Each dot represents a CpG site, with its vertical position indicating the negative logarithm (base 10) of the unadjusted p-value for DNA methylation association. The x-axis shows the relative log fold change (log FC) of the m-values between the two timepoints. Negative values indicate CpGs with decreased methylation among study participants (green), while positive values indicate increased methylation (red).

### Gene Ontology Pathways

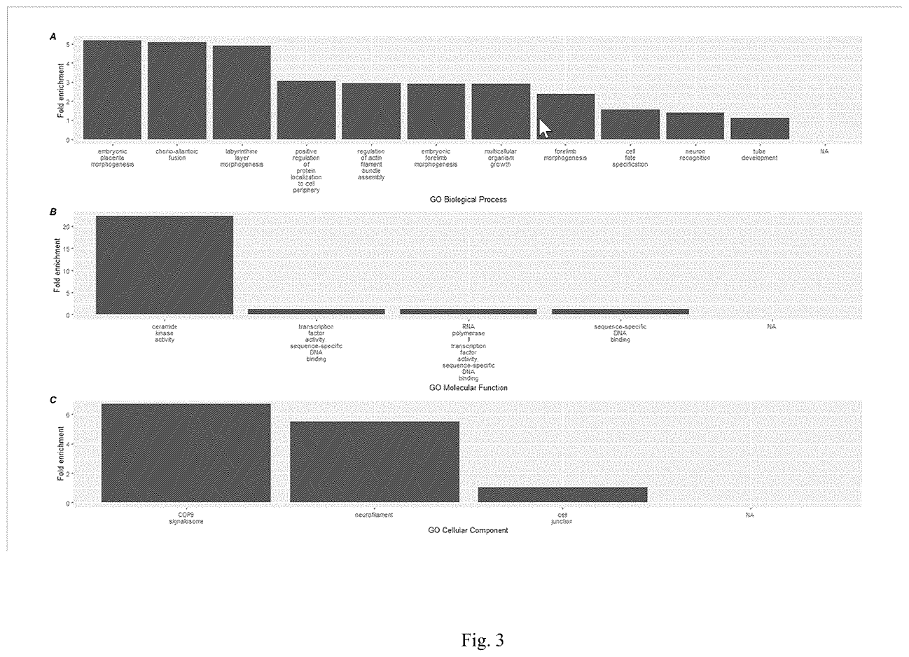

To link the methylation results to biological processes, enrichment analyses using the GREAT software were conducted on CpGs based on the direction of methylation and used to identify gene ontology (GO) pathways. Referring to FIG. 3, hypermethylated CpGs at the conclusion of the study were significantly associated with a total of (A) 15 GO-BP (Biological processes) terms, (B) 4 GO-MF (molecular function), and (C) 3 GO-CC (Cellular component) terms. The top 15 terms for each GO category included a diverse group of pathways including ceramide kinase activity, COP9 signalosome activity, labyrinthine layer morphogenesis, and neurofilament activity.

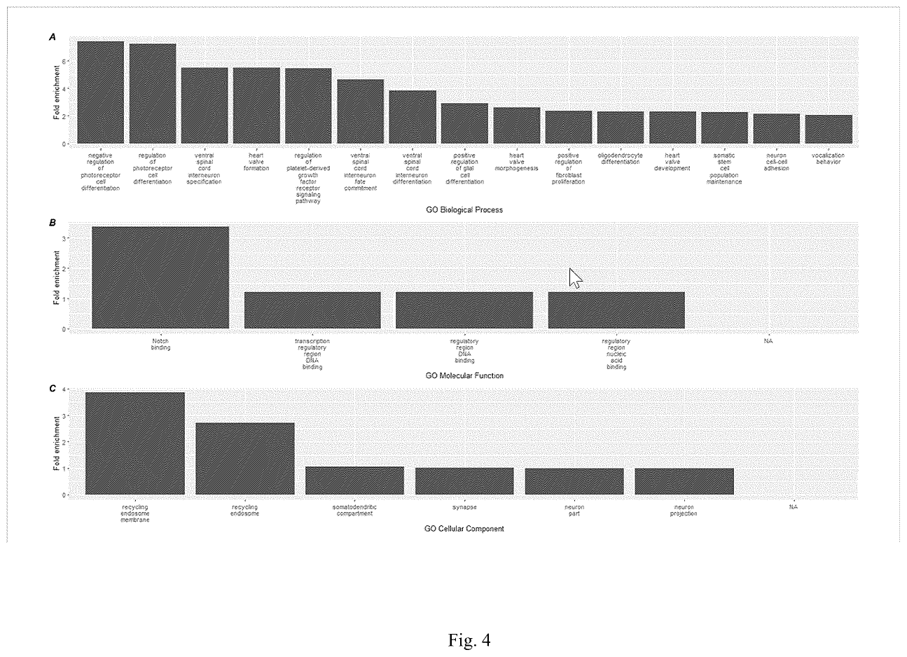

A similar analysis was performed on the hypomethylated DMLs identified in the analysis, which revealed greater significant GO terms compared to the hypermethylated DMLs. Among the hypomethylated DMLs, we determined 124 GO-BP terms, 6 GO-MF terms, and 4 GO-CC terms. The top 15 for each category are reported in FIG. 4 which includes the activation of processes that regulate photoreceptor cell differentiation and ventral spinal cord interneuron specification. In addition, we also observed higher enrichment of processes associated with Notch binding.

### Comparison to Published Dietary DML Data

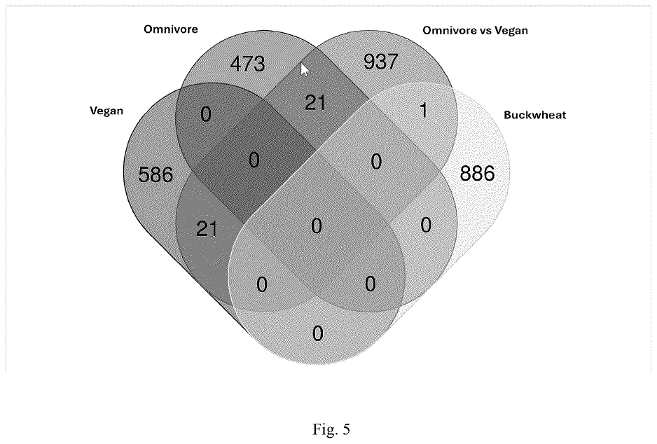

To better interrogate whether the above results might represent changes seen in more comprehensive dietary intervention, and whether the study supplement mimicked the effects of a plant-based diet, we compared our data to the results from Dwaraka et al. BMC medicine. (2024) 22:301., which examined DMLs across a vegan intervention and omnivore (control) intervention using a Venn diagram of DMLs across the three groups (FIG. 5).

The one CpG shared among the current analysis with the Omnivore analysis is cg05093714 (Gene ID: LINC01095) which is significantly higher in the vegan cohort compared to the omnivore cohort at 8 weeks. However, all other CpGs are specific to the Tartary buckwheat cohort identified here. No overlap is observed between the Vegan or Omnivore diet, suggesting different pathways are involved.

### Study Supplement Use Impacts Changes in Epigenetic Age

To determine the response to the study supplement on biological age, we quantified and performed analysis on a host of biological age metrics using DNA methylation. Aging clocks used included the second generation multi-omic informed OMICmAge, the third generation DunedinPACE (PACE) and principal component (PC) based second generation PhenoAge and GrimAge clocks. We additionally utilized epigenetic age acceleration (EAA), a marker of the difference between expected rate of aging based on chronological and biological aging.

Remarkable findings included:
1. A slowing of epigenetic age acceleration in people with a PCPhenoAge 1SD higher than the mean (p=0.031)
2. An increase in epigenetic age acceleration in people with a PCGrimAge 1SD lower than the mean (p=0.031)
3. An increase in epigenetic age acceleration in people with a OMICmAge 1SD lower than the mean (p=0.031).
The full results of this analysis are represented in Table 3.

TABLE 3
Investigation of epigenetic age measures based on subsets one standard deviation higher
than the mean, one standard deviation lower than the mean, and within or equal to one
standard deviation (−1 to +1) of the mean for multiple epigenetic aging algorithms.

| | Mean - | SD - | Mean - | Mean - SD | | Wilcoxon |
|---|---|---|---|---|---|---|
| | Test 1 | Test 1 | Test 2 | Test 2 | N | (p-value) |
| OMICmAge EAA - 1SD Higher | 5.686 | 1.176 | 4.185 | 2.904 | 7 | 0.380 |
| OMICmAge - 1SD Higher | 61.290 | 10.649 | 59.940 | 11.844 | 7 | — |
| OMICmAge EAA - 1SD Lower | −4.969 | 1.037 | −3.718 | 1.851 | 7 | 0.031 |
| OMICmAge - 1SD Lower | 48.032 | 5.557 | 49.404 | 5.709 | 7 | — |
| OMICmAge EAA - Within 1SD | −0.291 | 1.906 | −0.031 | 3.120 | 26 | 0.860 |
| OMICmAge - Within 1SD | 54.785 | 7.201 | 55.268 | 7.538 | 26 | — |
| OMICmAge EAA - All | −0.063 | 3.604 | 0.062 | 3.699 | 40 | 0.740 |
| OMICmAge - All | 54.741 | 8.439 | 55.059 | 8.548 | 40 | — |
| PCPhenoAge EAA - 1SD Higher | 8.065 | 2.163 | 4.262 | 2.294 | 6 | 0.031 |
| PCPhenoAge - 1SD Higher | 51.004 | 4.785 | 47.456 | 6.345 | 6 | — |
| PCPhenoAge EAA - 1SD Lower | −9.300 | 2.886 | −5.790 | 4.221 | 7 | 0.078 |
| PCPhenoAge - 1SD Lower | 36.754 | 11.490 | 40.537 | 11.347 | 7 | — |
| PCPhenoAge EAA - Within 1SD | −0.087 | 2.435 | 0.166 | 4.132 | 27 | 0.360 |
| PCPhenoAge - Within 1SD | 48.759 | 10.207 | 49.251 | 10.601 | 27 | — |
| PCPhenoAge EAA - All | −0.476 | 5.580 | −0.262 | 4.854 | 40 | 0.690 |
| PCPhenoAge - All | 46.995 | 10.778 | 47.457 | 10.522 | 40 | — |
| PCGrimAge EAA - 1SD Higher | 2.960 | 0.822 | 2.177 | 1.455 | 8 | 0.200 |
| PCGrimAge - 1SD Higher | 63.209 | 11.339 | 62.561 | 11.717 | 8 | — |
| PCGrimAge EAA - 1SD Lower | −3.759 | 0.554 | −1.333 | 0.536 | 6 | 0.031 |
| PCGrimAge - 1SD Lower | 57.527 | 12.238 | 60.078 | 12.357 | 6 | — |
| PCGrimAge EAA - Within 1SD | −0.407 | 1.165 | −0.336 | 2.166 | 26 | 0.670 |
| PCGrimAge - Within 1SD | 61.605 | 7.425 | 61.855 | 7.860 | 26 | — |
| PCGrimAge EAA - All | −0.236 | 2.248 | 0.017 | 2.178 | 40 | 0.320 |
| PCGrimAge - All | 61.314 | 8.979 | 61.730 | 9.187 | 40 | — |
| PACE - 1SD Higher | 1.007 | 0.050 | 0.976 | 0.071 | 8 | 0.250 |
| PACE - 1SD Lower | 0.710 | 0.033 | 0.699 | 0.090 | 7 | 0.690 |
| PACE - Within 1SD | 0.841 | 0.050 | 0.866 | 0.079 | 25 | 0.110 |
| PACE - All | 0.851 | 0.104 | 0.859 | 0.116 | 40 | 0.610 |

Italicized terms meet statistical significance at a p-value of 0.05 or less.

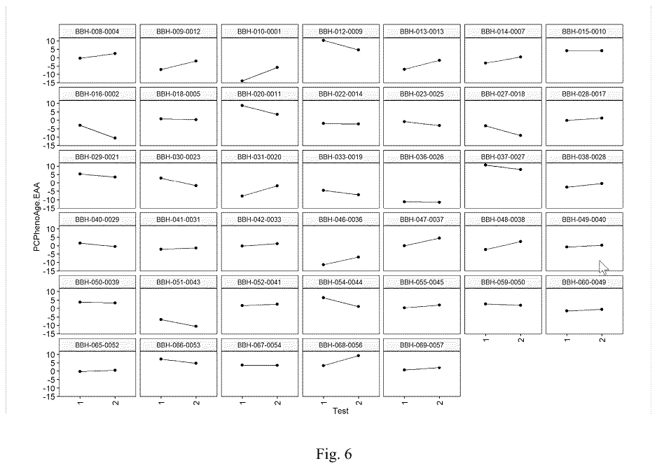

It is important to note the heterogeneity of individual results measured using age-related algorithms across the 90-day study period. An individual's epigenetic response to environmental factors and interventions may be significantly influenced by their pre-existing epigenetic status as well as a host of other factors known to impact epigenetic expression (e.g., gut microbiome composition, immune cell makeup, baseline exercise and dietary regimen). To this end, one would expect diversity in epigenetic age-related outcomes across participants. This can be visualized in FIG. 6 using data on the 40 study participants included in the final analysis. Positive slopes represent increased epigenetic age acceleration and negative slopes represent decreased epigenetic age acceleration across the 90-day study period.

### Deconvoluted Immune Cell Analysis

Alterations in immune cell makeup and function have been studied in references to both dietary change and specific nutrient augmentation. We used deconvolution methods to determine immune cell population changes over the duration of the study period to explore the effects of study supplementation on immune cell parameters across different biological age algorithms and within subsets of study participants. The PhenoAge algorithm has been independently validated to correlate with multiple markers of immunosenescence which are serologically determined. These include populations of T cells, B cells and granulocytes. To this end, we chose to apply the deconvolution methods to PCPhenoAge subgroupings. These can be reviewed in Table 3.

### Discussion of Results

In analysis of immune cell subtypes using deconvolution methods, the present inventors hypothesized the potential for immunosenescence related immune cell changes. Therefore, we ran the deconvolution methods against subset-specific data in Table 4 above for the PCPhenoAge evaluation. Notable here were significant increases in CD4 T memory cells (PCPhenoAge—1SD Higher) and CD8 T memory cells (PCPhenoAge—1SD Higher), significant decreases in B naive cells (PCPhenoAge—1SD Lower) and significant decreases in Natural Killer cells (PCPhenoAge—Within 1 SD). While the PCPhenoAge 1SD higher population saw a decrease in speed of epigenetic age progression, this group also demonstrated an increase in CD4T and CD8T memory cells. Conversely, those starting the study at a lower overall epigenetic age score saw a decrease in naïve B cells. These alterations in adaptive immune cells speak to potential effects on immune phenotypes over a 90-day interventional window. Additional and more comprehensive profiling of immune cell and cytokine alterations linked to Tartary buckwheat nutrient intake may better characterize the immune effects of dietary consumption of this seed, including on immunosenescence-related pathways. Using genome-wide EWAS analysis comparing blood samples from the start and end of the study period, we identified 887 differentially methylated CPG sites at a p value of <0.001 with 336 hypermethylated and 551 hypomethylated sites controlled for potential overfitting. These differentially methylated sites were then analyzed using the GREAT software to identify GO pathways. When we mapped CpG methylation against known biological processes using the GREAT software, a total of 22 pathways were linked to hypermethylated CpGs, while 134 were linked to hypomethylated CpGs. On review of these processes, the most highly enriched changes in biological pathways occurred within the hypermethylated CpG sites, where the largest fold enrichment (22×) was linked to ceramide kinase activity. We additionally found a 6.7-fold enhancement in COP9 activity. Among the hypomethylated biological processes, the largest enrichment changes were seen with negative and positive regulation of photoreceptor cell differentiation (7.41 and 7.21-fold, respectively). While of lower overall effect magnitude, it is also notable in the context of immunity that positive regulation of glial cell maturation was among the top 10 most pronounced findings, at a 2.9× fold-enrichment. These relatively large effects, in contrast with the immune cell deconvolution results, suggest that the more pronounced impact of the dietary supplementation occurred on upstream immune-related biological pathways measurable through epigenetics.

TABLE 4
Representation of changes in deconvoluted immune cell subsets
using epigenetic age accelerations for PCPhenoAge

| | Week | Week | Wilcoxon - | |
|---|---|---|---|---|
| | 0 | 8 | p-value | Clock comparison |
| CD4Tmem | 0.096 | 0.095 | 0.934 | PCPhenoAge - Within |
| CD8Tmem | 0.053 | 0.052 | 0.427 | PCPhenoAge - Within |
| CD4Tnv | 0.073 | 0.077 | 0.117 | PCPhenoAge - Within |
| CD8Tnv | 0.034 | 0.032 | 0.178 | PCPhenoAge - Within |
| Bmem | 0.020 | 0.019 | 0.645 | PCPhenoAge - Within |
| Bnv | 0.042 | 0.040 | 0.470 | PCPhenoAge - Within |
| Treg | 0.008 | 0.009 | 0.427 | PCPhenoAge - Within |
| Baso | 0.019 | 0.018 | 0.594 | PCPhenoAge - Within |
| Eos | 0.012 | 0.009 | 0.786 | PCPhenoAge - Within |
| NK | 0.060 | 0.052 | 0.046 | PCPhenoAge - Within |
| Neu | 0.521 | 0.540 | 0.441 | PCPhenoAge - Within |
| Mono | 0.063 | 0.057 | 0.220 | PCPhenoAge - Within |
| CD4Tmem | 0.105 | 0.087 | 0.156 | PCPhenoAge - 1SD Lower |
| CD8Tmem | 0.059 | 0.045 | 0.219 | PCPhenoAge - 1SD Lower |
| CD4Tnv | 0.143 | 0.118 | 0.109 | PCPhenoAge - 1SD Lower |
| CD8Tnv | 0.055 | 0.039 | 0.078 | PCPhenoAge - 1SD Lower |
| Bmem | 0.019 | 0.017 | 0.297 | PCPhenoAge - 1SD Lower |
| Bnv | 0.067 | 0.055 | 0.031 | PCPhenoAge - 1SD Lower |
| Treg | 0.013 | 0.011 | 0.469 | PCPhenoAge - 1SD Lower |
| Baso | 0.018 | 0.014 | 0.016 | PCPhenoAge - 1SD Lower |
| Eos | 0.007 | 0.003 | 0.402 | PCPhenoAge - 1SD Lower |
| NK | 0.063 | 0.057 | 0.469 | PCPhenoAge - 1SD Lower |
| Neu | 0.398 | 0.512 | 0.109 | PCPhenoAge - 1SD Lower |
| Mono | 0.052 | 0.042 | 0.375 | PCPhenoAge - 1SD Lower |
| CD4Tmem | 0.071 | 0.107 | 0.031 | PCPhenoAge - 1SD Higher |
| CD8Tmem | 0.037 | 0.855 | 0.031 | PCPhenoAge - 1SD Higher |
| CD4Tnv | 0.044 | 0.063 | 0.063 | PCPhenoAge - 1SD Higher |
| CD8Tnv | 0.034 | 0.042 | 0.156 | PCPhenoAge - 1SD Higher |
| Bmem | 0.014 | 0.020 | 0.031 | PCPhenoAge - 1SD Higher |
| Bnv | 0.032 | 0.039 | 0.156 | PCPhenoAge - 1SD Higher |
| Treg | 0.007 | 0.005 | 0.438 | PCPhenoAge - 1SD Higher |
| Baso | 0.014 | 0.019 | 0.156 | PCPhenoAge - 1SD Higher |

### Conclusion

These data demonstrate the potential for phytochemical nutrients found within Himalayan Tartary buckwheat to act on multiple metrics of epigenetic immune age, immune cells and related cellular pathways. The polyphenol-rich supplement designed around key bioactive nutrients naturally occurring in Tartary buckwheat appeared to directionally influence CpG methylation patterns in subsets of participants with higher and lower rates of biological aging as measured by the PCPhenoAge, PCGrimAge and OmicAge aging algorithms. These changes were correlated with changes in peripheral immune cell patterns as measured by the PCPhenoAge aging algorithm which is known to be sensitive to changes associated with immunosenescence. Changes in GO pathways additionally suggest the potential for effects on multiple immune and cellular regulatory mechanisms, especially those related to ceramide kinase. These results suggest that polyphenols and associated bioactive nutrients naturally occurring in Tartary buckwheat may significantly influence epigenetic age measurements that may be driven by or reflected in changes in the immune system and associated pathways.

### 4. CHARTS

Chart 1: Analysis of antioxidant and polyphenol composition of Tartary buckwheat flour and sprouted Tartary buckwheat flour showing changing trend of the flavonoid and chlorophyll contents during the gemination of Tartary buckwheat.
Chart 2:
Chart 3: The changing trend of the flavonoid and chlorophyll contents during the germination of tartary buckwheat.

Source: (Evolution of nutrient ingredients in tartary buckwheat seeds during germination)

### 5. INDUSTRIAL APPLICABILITY

### 1. Commercial Applications

The invention leveraging Tartary buckwheat's unique bioactive profile to combat inflammaging has broad and diverse commercial applications across several industries:
Functional Foods:
   Tartary buckwheat can be incorporated into a range of products, such as breakfast cereals, granola bars, snacks, and meal replacements, targeting health-conscious consumers.
   Enhanced formulations tailored for aging populations or individuals with specific health conditions (e.g., joint health, heart health).
Nutraceuticals:
   Tartary buckwheat extracts, rich in flavonoids like rutin and quercetin, can be standardized and formulated into capsules, tablets, or powders for daily supplementation.
   Marketed for managing inflammation, oxidative stress, and aging-related conditions.
Beverages:
   Ready-to-drink formulations, such as functional teas, smoothies, or energy drinks, incorporating Tartary buckwheat extracts or powders.
   Positioned in the growing market for natural and plant-based health drinks.
Cosmeceuticals:
   Tartary buckwheat polyphenols can be integrated into anti-aging skincare formulations to provide antioxidant and anti-inflammatory benefits, addressing skin inflammaging.
Medical Nutrition:
   Products designed for clinical use, such as supplements for patients recovering from inflammatory conditions or for preventive care in aging individuals.

### 2. Scalability

The scalability of this invention is supported by the following factors:
Agricultural Availability:
   Tartary buckwheat is a resilient crop that can be cultivated in diverse climatic conditions, including marginal lands unsuitable for other crops. This ensures a sustainable and cost-effective supply chain.
Processing Innovations:
   Modern agricultural and food processing techniques, such as micronization and extraction technologies, enhance the bioavailability and stability of active compounds.
   Fermentation and other value-addition processes can improve the sensory qualities and nutritional value of Tartary buckwheat-based products.
Global Demand for Natural Products:
   Growing consumer preference for natural, plant-based health solutions creates a robust market opportunity for Tartary buckwheat-derived products.
   The demand for gluten-free and allergen-free food alternatives further expands its market potential.

### 3. Market Potential

The invention aligns with several rapidly growing market segments, ensuring substantial commercial viability:
Anti-aging and Inflammaging Markets:
   The global anti-aging market is projected to reach over $400 billion by 2030, with increasing demand for solutions targeting the root causes of aging, such as chronic inflammation.
   Tartary buckwheat-derived products can be uniquely positioned to address inflammaging, differentiating them from conventional anti-aging supplements.
Functional Food and Beverage Market:
   The functional foods market is expected to grow at a CAGR of over 8% in the coming years, driven by consumer interest in preventative health solutions.
   Products containing Tartary buckwheat can cater to this demand by emphasizing their scientifically validated health benefits.
Nutraceuticals Market:
   The global nutraceutical market, currently valued at over $400 billion, offers immense potential for Tartary buckwheat supplements targeting inflammation, oxidative stress, and metabolic health.
Emerging Consumer Trends:
   Interest in clean-label, sustainable, and functional ingredients positions Tartary buckwheat as a desirable option for innovative product development.

What is claimed is:

1. The process of inhibiting biologic aging in human or domesticated animal subject by certain administration steps of Tartary buckwheat utilizing enhanced amounts of polyphenol-rich agents administered in dosages effective for particular categories of subjects to elevate immune function through epigenetic alteration by steps of:
(a) creating an enhanced form of Tartary buckwheat by cold environment seed or root planting harvested at post germination sprouting stage and adding beneficial microorganisms at seeding and/or growth stages pre-sprouting guided by methylation patterns;
(b) micronization processing of the harvested material to powder, leaf capsules or liquid forms for ingestion in diet and/or blood system inoculation, the beneficial microorganisms being selected from the group consisting of symbiotic bacterial, mycorrhizal fungi, singly or in combinations, thereof;
(c) further adding at sprout stage or subsequently in dosing in diet and/or inoculation additives selected from the group of omega-3 fatty acids, probiotics, prebiotics, vitamins, minerals, fermented products or other nutraceuticals, singly and/or any combination;
(d) adjusting diet or inoculation of a subject in relation to one or more singly or any combination markers from the group consisting of a subject's genomic instability, telomere alteration, epigenetic alteration, loss of proteostasis, deregulated nutrient sensing, mitochondrial, dysfunction, cellular senescence, stem cell, exhaustion, altered intercellular communication;
(e) managing over a selected term of a group consisting of a program of such diet and/or inoculation effects and if indicated revising the program to measure improvement of immune function, biological aging through increased epigenetic alterations through a pattern of CpG alterations and/or inhibition of pro-inflammatory cytokines including IL-6, TNF-alpha or beneficial alterations in the immune system including positive changes to innate or adaptive immune cell makeup.

2. The process of claim 1 wherein after seed planting and germination in cold stress drought conditions, the sprouting is done at a stage effective to produce content of rutin in the sprouted Tartary buckwheat of more than 100 times or more versus content of unsprouted buckwheat and quercetin content of 90 times or more versus content of unsprouted buckwheat and wherein the polyphenol content is 2 fold compared to unsprouted common buckwheat.

3. The process of claim 2 wherein the harvesting is done 3 days after germination and the milling or other micronization step is done 4 days after initial sprouting.

4. The process of claim 3 wherein fermentation is combined with micronization.

5. The composition of processed Tartary buckwheat as produced by the process of claim 1.

6. The process of feeding subjects the composition of claim 5 approximately/preferably at the amounts shown on Table 1 Formulation of the specification for a period of 12 to 24 weeks, without prejudice to benefits of feeding at lesser or greater time durations for subjects with population characteristics as shown on Table 2 of Formulation 1.

7. The process of claim 6 adjusted to achieve by demethylation as indicated in FIGS. 1-3 and related specification text and ultimate results as shown on Tables 3 and 4.

8. The process of reducing elderly age related immunosenescence and/or chronic inflammation and related disorders thorough dietary regular comprising use of dietary polyphenols uniquely abundant in Tartary buckwheat created, processed and adjusted as set forth in claim 1.
